(19) United States
(12) Patent Application Publication
(10) Pub. No.: US 2024/0416048 A1
(43) Pub. Date: Dec. 19, 2024

(54) DELIVERY MEMBER COVER

(71) Applicant: SHL Medical AG, Zug (CH)

(72) Inventors: Yen-Chun Chen, Taoyuan City (TW); Daniel Carlsson, Nacka Strand (SE); Daniel Säll, Nacka Strand (SE)

(21) Appl. No.: 18/702,937

(22) PCT Filed: Oct. 7, 2022
(86) PCT No.: PCT/EP2022/077940

§ 371 Date: Apr. 19, 2024

(30) Foreign Application Priority Data
Oct. 21, 2021 (EP) 21204010.9

(51) Int. Cl. A61M 5/32

(52) U.S. Cl. CPC A61M 5/3204; A61M 2005/3267

(57) ABSTRACT

The present disclosure provides a delivery member cover of a medicament delivery device. The delivery device includes a medicament container structure having a delivery member for expelling a medicament. The delivery member cover is configured to cover at least the delivery member. The delivery member cover includes housing structure having a proximal end, a distal end, and a receiving recess arranged between the proximal end and the distal end. The receiving recess is configured to receive at least a portion of the medicament container structure. The delivery member cover further includes a resilient portion arranged inside the receiving recess. The resilient portion is configured to flex radially outwards to radially support said portion of the medicament container structure upon insertion of the medicament container structure into the receiving recess.

### CROSS-REFERENCE TO RELATED APPLICATIONS

The present application is a U.S. National Phase Application pursuant to 35 U.S.C. § 371 of International Application No. PCT/EP2022/077940 filed Oct. 7, 2022, which claims priority to European Patent Application No. 21204010.9 filed Oct. 21, 2021. The entire disclosure contents of these applications are herewith incorporated by reference into the present application.

### TECHNICAL FIELD

The present disclosure generally relates to a medicament delivery device for expelling medicament from a flexible medicament container, and in particular to a delivery member cover of a medicament delivery device.

### BACKGROUND

A number of medical conditions require reliable provision of a medicament from a medicament delivery device. The medicament is typically comprised in a medicament container within the medicament delivery device, the medicament container being configured to expel the medicament via some type of delivery member, such as a needle or a nozzle.

These days, a number of different medicament delivery devices exist, including various types of injectors (for example pen injectors, autoinjectors, on-body devices). Although many of these devices have enabled major improvements in the management of a number of medical conditions, various drawbacks do still exist in the current technology.

For example, the medicament delivery device often comprises some type of delivery member cover configured to cover the delivery member prior to a medicament delivery operation, as well as subsequent to such medicament delivery operation. For the latter, the medicament container is typically held in a retracted position within the medicament delivery device, while the delivery member, e.g. a needle, is prevented from being exposed in order avoid accidental re-use of the device. Thus, the delivery member is configured to move relative the delivery member cover. In order to facilitate this relative movement, a play is typically present between the medicament container and inner portions of the delivery member cover. Such play may give rise to a rattling sound, in particular in the retracted position of the medicament container. In considering these problems, the applicant has appreciated that various developments could be made to help improve the medicament delivery devices on the market today, which are set out in more detail below.

### SUMMARY

An object of the present disclosure is thus to provide a medicament delivery device, and in particular a delivery member cover for a medicament delivery device which solves, or at least mitigates, problems of the prior art.

There is hence according to a first aspect of the present disclosure provided a delivery member cover of a medicament delivery device, the delivery device comprising a medicament container structure having a delivery member for expelling a medicament, the delivery member cover being configured to cover at least the delivery member, the delivery member cover comprising: a housing structure having a proximal end, a distal end and a receiving recess arranged between the proximal end and the distal end, the receiving recess being configured to receive at least a portion of the medicament container structure; a resilient portion arranged inside the receiving recess, the resilient portion being configured to flex radially outwards to radially support said portion of the medicament container structure upon insertion of the medicament container structure into the receiving recess.

An effect obtainable thereby is that improved support of the medicament container structure may be provided. Thus, rattling noise caused by the medicament container structure as it is housed in the receiving recess of the delivery member cover may be reduced, or even avoided.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component. Similarly, the terms “transverse”, “transversal” and “transversally” refer to a direction generally perpendicular to the longitudinal direction. Further, the terms “circumference”, “circumferential”, “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to one embodiment the resilient portion is radially movable within the receiving recess between a first position in which the resilient portion defines a first cross section area of the receiving recess, and a second position in which the resilient portion defines a second cross section area of the receiving recess, wherein the second cross section area is larger than the first cross section area. The resilient portion is thus arranged movable from the first position to the second position to thereby enable the receiving recess to be enlarged, at least by providing a larger cross section area. The cross section area may be the diameter of the receiving recess at the position of the resilient portion. Thus, the first cross section area may be referred to as a first diameter, and the second cross section area may be referred to as a second diameter.

According to one embodiment, the first cross section area in the first position of the resilient portion is smaller than a cross section area of said portion of the medicament container structure. Hereby, upon insertion of the medicament container structure into the receiving recess, the resilient portion is ensured to be brought into contact with the medicament container structure.

According to one embodiment, the resilient portion is configured to be displaced from the first position to the second position upon insertion of the medicament container structure into the receiving recess.

The resilient portion is typically configured to engage with said portion of the medicament container structure in such a way that the resilient portion radially moves from the first position to the second position. Stated differently, the receiving recess is sized and dimensioned to receive and house the medicament container structure, such that, upon insertion of the medicament container structure into the receiving recess, the resilient portion is arranged to move from its first position to its second position.

Thus, as the cross section area, or diameter, of said portion of the medicament container structure is larger than the first cross section area of the receiving recess, said portion of the medicament container structure will, upon insertion of said portion of the medicament container structure into the receiving recess, push the resilient portion to radially move from its first position to its second position. Preferably, at the second position of the resilient portion, the second cross section area of the receiving recess is the same, or substantially the same, as the cross section area of said portion of the medicament container structure.

According to one embodiment, the resilient portion is configured to bear against said portion of the medicament container structure upon insertion of the medicament container structure into the receiving recess. Hereby, the said portion of the medicament container structure is supported by the resilient portion.

According to one embodiment, the resilient portion comprises a contact surface configured to be in contact with said portion of the medicament container structure upon insertion of the medicament container structure into the receiving recess, wherein the contact surface comprises a thermoplastic elastomer. Hereby, the support of the medicament container structure may be further improved.

According to one embodiment, the resilient portion is arranged between the proximal end and the distal end of the housing structure. Such positioning of the resilient portion is advantageous, as the support for the medicament container structure is directed at least to a position between the proximal end and the distal end of the housing structure. According to one embodiment, the resilient portion is arranged in a mid-portion of the housing structure, the mid-portion composing 50% of the longitudinal extension of the housing structure equally distant from the distal end and the proximal end of the housing structure.

According to one embodiment, the resilient portion forms a part of the housing structure. The resilient portion may be formed out of the same material as the housing structure, e.g. plastic. The resilient portion may e.g. be arranged on a housing portion facing the receiving recess.

According to one embodiment, the resilient portion extends from an outer surface of the housing structure into the receiving recess. For example, the resilient portion may comprise a protrusion of the housing structure extending from an outer surface into the receiving recess. Alternatively, the resilient portion extends from an external surface of the housing structure into the receiving recess. The external surface may be a surface of the housing structure facing inwards (i.e. an inner surface), or it may be the outer surface facing outwards. According to one embodiment, the resilient portion may present a decreasing ramp surface in the distal direction. The resilient portion may be formed as a ramp extending into the receiving recess, wherein the ramp surface decrease in the distal direction to facilitate interaction with said portion of the medicament container structure. Stated differently, the ramp surface is angled in the distal direction so that it faces towards the centre axis and towards the distal end. Thus, a first end of the ramp surface is arranged closer to the centre axis compared to a second end of the ramp surface, wherein the ramp surface axially extends from the first end to the second end, and wherein the first end is arranged proximal of the second end. Hereby, the resilient portion may flex radially outwards as it engages with said portion of the medicament container structure upon insertion of the medicament container structure into the receiving recess. The resilient portion may alternatively be a tongue, a tab, or an arm extending from the housing structure into the receiving recess. The resilient portion typically extends radially and longitudinally into the receiving recess.

According to one embodiment, the housing structure comprises a counteracting surface arranged opposite to the resilient portion, wherein the housing structure is configured to receive said portion of the medicament container structure between the resilient portion and the counteracting surface. The counteracting surface is typically a surface in a portion of the housing structure facing the receiving recess. Thus, the previously mentioned first and second cross section areas (or diameters) may be defined by the resilient portion and the counteracting surface. The counteracting surface may form a continuous portion of the inner facing surface of the housing structure, and may thus not be resilient.

According to one embodiment, the resilient portion is a first resilient portion and the delivery member cover further comprises a second resilient portion arranged on an opposite side of the housing structure compared to first resilient portion, wherein the housing structure is configured to receive said portion of the medicament container structure between the first and second resilient portions. Hereby, the support of the medicament container structure may be further improved. Thus, the previously mentioned counteracting surface may be comprised in said second resilient portion.

According to one embodiment, the resilient portion are multiple resilient portions configured to receive said portion of the medicament container structure between the multiple resilient portions. Thus, the delivery member cover may be provided with more than one, or more than two resilient portions configured to engage with, and radially support, said portion of the medicament container structure upon insertion of the medicament container structure into the receiving recess.

According to one embodiment, the first resilient portion and/or the second resilient portion is a tab and/or the multiple resilient portions are tabs. Such configuration of the resilient portion is advantageous as a tab is a relatively simple structure, which can be made flexible or resilient. The at least one tab may be referred to as an inner tab arranged on a surface of the housing structure facing the receiving recess.

According to one embodiment, the delivery member cover is tubular, or is at least partly formed of a tubular portion. The tubular portion of the delivery member cover may e.g. extend along a majority of the longitudinal extension of the delivery member cover. The housing structure of the delivery member cover typically comprises an outer sleeve having an external surface facing outwards (i.e. an outer surface). The housing structure of the delivery member cover typically comprises an inner sleeve having an external surface facing inwards (i.e. an inner surface). Thus, the housing structure of the delivery member cover may be tubular, or may be at least partly formed of a tubular portion.

According to one embodiment, the delivery member cover comprises a first tubular portion arranged at the proximal end and a second tubular portion arranged distally of the first tubular portion. Typically, the second tubular portion has a smaller cross section area, or smaller inner diameter, than the cross section area, or inner diameter, of the first tubular portion. Preferably, the resilient portion is arranged in the second tubular portion.

According to one embodiment, the resilient portion is a resilient structure. The resilient structure may form a part of the housing structure, or the resilient structure may form a separate unit to the housing structure, e.g. by being comprised in a coil spring arranged in the receiving recess of the delivery member cover.

There is according to a second aspect of the present disclosure provided a medicament delivery device comprising a medicament container structure having a delivery member for expelling a medicament, and a delivery member cover according to the first aspect of the present disclosure.

Effects and features of the second aspect of the invention are largely analogous to those described above in connection with the first aspect of the invention. Embodiments mentioned in relation to the first aspect of the invention are largely compatible with the second aspect of the invention, of which some are exemplified below.

According to one embodiment, the delivery member cover is configured to move axial relative to the delivery member from a delivery position in which the delivery member is uncovered for expelling the medicament, into a storage position in which the delivery member is covered by the delivery member cover.

According to one embodiment, the resilient portion of the delivery member cover is configured to radially support the medicament container structure while allowing axial movement of the medicament container structure within the delivery member cover. That is, the resilient portion is configured to allow axial movement of the medicament container structure within the delivery member cover, e.g. during insertion of the medicament container structure into the receiving recess of the delivery member cover, but also as the delivery member cover moves axial relative to the delivery member from the delivery position into the storage position.

Typically, the medicament container structure is a syringe, and the delivery member is a needle. Thus, the delivery member cover may be referred to as a needle cover. However, the invention is also applicable to other medicament container structures in which the delivery member is e.g. a nozzle, a vacuum injector or a high-pressure injector. The medicament delivery device may e.g. be an autoinjector.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 schematically shows a cross-sectional view of an example of a medicament delivery device comprising a medicament container structure and a delivery member cover.

FIG. 2 shows a perspective view of the delivery member cover in FIG. 1;

FIGS. 3A is sectional view of the delivery member cover of FIG. 2 during insertion of the medicament container structure into the receiving recess of the delivery member cover;

FIG. 3B is a sectional view of the delivery member cover of FIG. 2 as the medicament container structure has been inserted into the receiving recess of the delivery member cover;

FIGS. 4A-4B are perspective views of an alternative embodiment of the delivery member cover;

FIG. 5 shows a cross-sectional view of yet another example of a medicament delivery device comprising a medicament container structure and a delivery member cover, and an enlarged view of an alternative embodiment of the delivery member cover.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

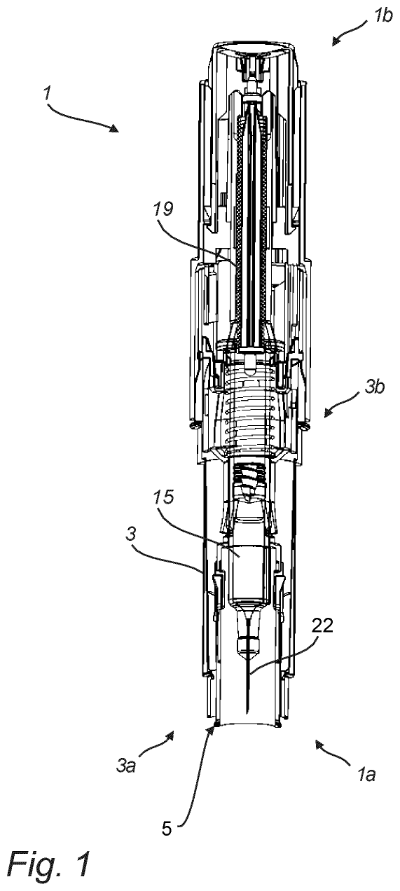

FIG. 1 shows an example of a medicament delivery device 1 configured to expel medicament from a medicament container structure 15. The medicament container structure 15 comprising a delivery member 22 for expelling the medicament. The medicament container structure 15 may for example be a syringe, and the delivery member 22 a needle.

The medicament delivery device 1 extends in the longitudinal direction from a proximal end la to a distal end 1b. The medicament delivery device 1 comprises a housing 3. Correspondingly, the housing 3 comprises a proximal end 3a and a distal end 3b.

The medicament delivery device 1 comprises a delivery member cover 5. The delivery member cover 5 is arranged in the housing 3 and at least a portion of the delivery member cover 5 extends proximally from the proximal end 3a of the housing 3.

The delivery member cover 5 is configured to be moved axially (or linearly) relative to the housing 3 from a first state to a second state in which the delivery member cover 5 is received further in the housing 3. In the second state, the delivery member 22 is uncovered for expelling a medicament. Thus, in the second state, the delivery member cover 5 may be referred to as a being in a delivery position. Subsequent to expelling the medicament, the delivery member cover 5 may be brought into a third state, or into a storage position, in which the delivery member 22 is covered by the delivery member cover 5. The delivery member cover 5 is typically biased in the proximal direction towards the first state.

In the following sections, an example embodiment describing the general structure and operation of the medicament delivery device 1 of FIG. 1 will be exemplified and described, and then, details and example embodiments of the delivery member cover 5 will be described.

The medicament delivery device 1 may be triggered to discharge medicament, or at least triggered to allow activation, by movement of the delivery member cover 5 further into the housing 3 towards the second state, or delivery position. For example, the medicament delivery device 1 may comprises a biased hollow drive member or expulsion member 19, such as a plunger rod 19. The drive member 19 may be configured to be moved axially inside the housing 3 upon activation. Such activation may e.g. be achieved by an actuator arranged in the distal end 1b of the medicament delivery device 1, or by activation upon movement of the delivery member cover 5 into its second state. During such medicament delivery operation, the drive member 19 is typically configured to be moved from a first axial position towards the proximal end 3a of the housing 3. For providing the biased condition, a spring, such as a coil spring, may be arranged inside the medicament delivery device 1. For example, when the delivery member cover 5 is brought into its second state, the drive member 19 is released, or is enabled to be released by activating the actuator, from its biased condition. For example, the drive member 19 may be held in its biased condition by means of a holding sleeve. The holding sleeve may be displaced to release the drive member from its biased condition by means of the actuator and/or by means of movement of the delivery member cover 5. Thus, according to one example, when the delivery member cover 5 is moved from the first state towards the second state, linear movement of the delivery member cover 5 is transformed into the holding sleeve, which moves to release the drive member 19. According to some variations, the drive member 19 could for example be motor driven or manually driven instead of having a spring-loaded structure.

After the medicament delivery operation, the delivery member cover 5 is configured to move axially relative to the delivery member 22, from the second state, or delivery position, into the third state, or a storage position, in which the delivery member 22 is covered by the delivery member cover 5. Thus, the medicament delivery device 1 may be disposed of in this final state, in which the delivery member 22 is protected inside the delivery member cover 5.

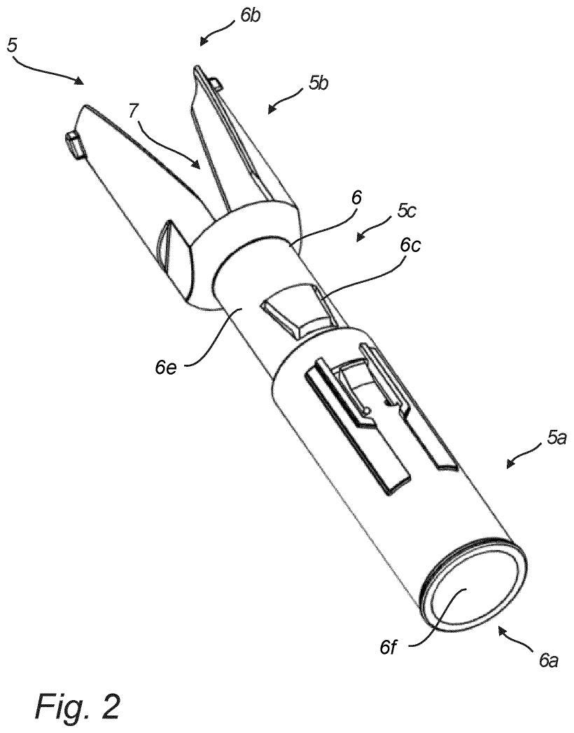

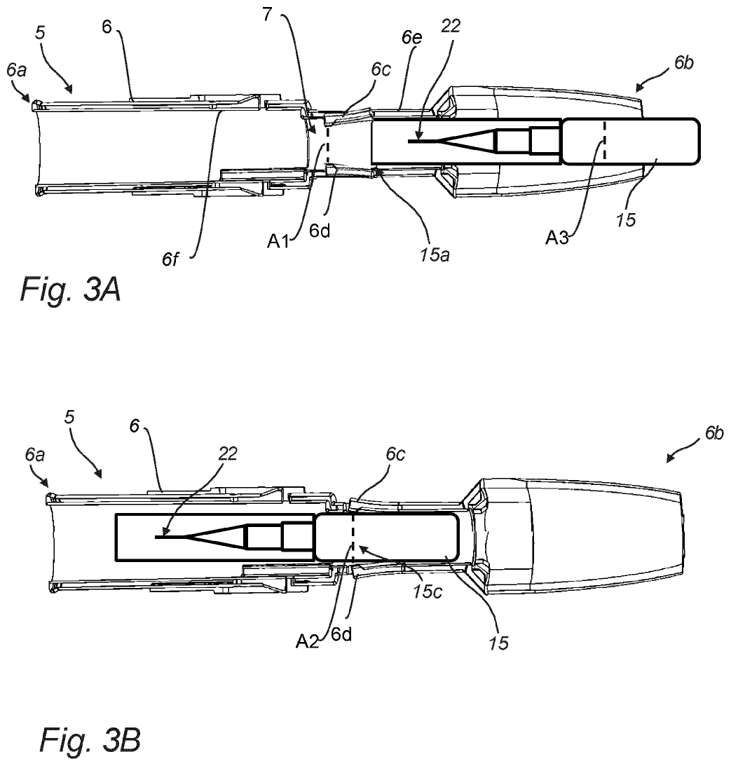

Turning to FIG. 2 and FIGS. 3A-3B, showing the delivery member cover 5 of the medicament delivery device 1 of FIG. 1. The delivery member cover 5 is configured to cover at least the delivery member 22 as described with reference to FIG. 1. The delivery member cover 5 comprises a housing structure 6 having a proximal end 6a, a distal end 6b and a receiving recess 7 arranged between the proximal end 6a and the distal end 6b. The delivery member cover 5 is tubular, or shaped as a hollow cylinder. As seen in FIG. 2, the shape of the delivery member cover 5 need not to be uniform, but different portions along the longitudinal direction may be shaped differently, e.g. by that the different portions have different diameters, or by that a portion is slitted (i.e. partly opened in the longitudinal direction) as seen at the distal end of the delivery member cover 5. Thus, at least a portion of the delivery member cover 5 may be formed by another shape than the tubular shape. In more detail, the delivery member cover 5 of FIG. 2 comprises a first tubular portion 5a arranged at the proximal end of the delivery member cover, a slitted portion 5b arranged at the distal end of the delivery member cover, and a second tubular portion 5c arranged between the first tubular portion 5a and the slitted portion 5b. Typically, the second tubular portion 5b has a smaller cross section area, or smaller inner diameter, than the cross section area, or inner diameter, of the first tubular portion 5a. However, according to one example embodiment, the first tubular portion 5a is arranged to have a smaller cross section area, or smaller inner diameter, than the cross section area, or inner diameter, of the second tubular portion 5b. The slitted portion 5b in FIG. 2 is slitted in the longitudinal direction, i.e. the portion comprises slits or openings along the longitudinal direction separating two distally extending elongations. According to at least one alternative embodiment, the delivery member cover 5 is of another shape than the tubular shape.

As seen in FIG. 2, the housing structure 6 comprises an outer sleeve having an external surface 6e, or an outer surface 6e, facing outwards. Moreover, the housing structure 6 comprises an inner sleeve having an external surface 6f, or an inner surface 6f, facing inwards. The receiving recess 7 is defined by the inner facing surface 6f, and is cylindrically shaped. In other words, the receiving recess 7 compose the hollow interior of the tubular delivery member cover 5. The receiving recess 7 is configured to receive at least a portion of the medicament container structure 15, as shown in FIGS. 3A-3B.

The delivery member cover 5 comprises at least one resilient portion 6c, 6d arranged between the proximal end 6a and the distal end 6b of the housing structure 6. Preferably, the at least one resilient portion 6c, 6d is arranged in the second tubular portion 5c. As shown in FIG. 3A, the delivery member cover 5 comprises a first resilient portion 6c and a second resilient portion 6d arranged on an opposite side of the housing structure 6 compared to first resilient portion 6c. Each one of the first and second resilient portions 6c, 6d forms a part of the housing structure 6. In more detail, each one of the first and second resilient portions 6c, 6d extends from the outer surface 6e of the housing structure 6 into the receiving recess 7. Each one of the first and second resilient portions 6c, 6d may extend into the receiving recess 7 at various different angles. For example, each one of the first and second resilient portions 6c, 6d may extend substantially perpendicular from the outer surface 6e or inner surface 6f into the receiving recess 7. According to another example, each one of the first and second resilient portions 6c, 6d extend from the outer surface 6e or inner surface 6f into the receiving recess 7 at an angle of 45° or less. Thus, each one of the first and second resilient portions 6c, 6d may extend both radially and longitudinally into the receiving recess 7. Thus, the first and the second resilient portions 6c, 6d are arranged inside the receiving recess 7. Moreover, each one of the first and second resilient portions 6c, 6d is arranged as a tab inside the receiving recess 7.

The resilient portions 6c, 6d are resilient, meaning that they are flexibly arranged within the receiving recess 7. In other words, each one of the resilient portions 6c, 6d is radially movably within the receiving recess 7. The first and the second resilient portions 6c, 6d are movable between a respective first position in which the first and second resilient portions 6c, 6d define a first cross section area A1 of the receiving recess 7 (which e.g. is defined by a first diameter or a first distance between the first and second resilient portions 6c, 6d, such as the shortest distance between the first and second resilient portions 6c, 6d at their first position), and a respective second position in which the first and second resilient portions 6c, 6d define a second cross section area A2 of the receiving recess 7 (which e.g. is defined by a second diameter or a second distance between the first and second resilient portions 6c, 6d, such as the shortest distance between the first and second resilient portions 6c, 6d at their second position). The first position of the resilient portions 6c, 6d and the first cross section area A1 are shown in FIG. 3A, and the second position of the resilient portions 6c, 6d and the second cross section area A2 are shown in FIG. 3B. As clearly shown by comparing the position of the first and second resilient portions 6c, 6d in FIGS. 3A-3B, the second cross section area A2 is larger than the first cross section area A1. The insertion of the medicament container structure 15 into the receiving recess 7 will now be described with reference to FIGS. 3A-3B. As shown in FIG. 3A, the insertion of the medicament container structure 15 has begun, and a proximal portion 15a of the medicament container structure 15 (here being an RNS) has reached a position within the delivery member cover 5 just prior, or adjacent, the first and second resilient portions 6c, 6d. Here, the first and second resilient portions 6c, 6d are still in their respective first position.

As the medicament container structure 15 is moved further in the proximal direction, the medicament container structure 15 reaches, and at least partly passes, the cross section of the receiving recess being defined by the first and second resilient portions 6c, 6d. The first and second resilient portions 6c, 6d are configured to flex radially, and as the medicament container structure 15 moves at least partly pass the first and second resilient portions 6c, 6d, the first and second resilient portions 6c, 6d flex radially outwards. Thus, the first and second resilient portions 6c, 6d are configured to be displaced from their respective first position to their respective second position upon insertion of the medicament container structure 15 into the receiving recess 7.

As the medicament container structure 15 reaches its final position within the receiving recess 7, a portion 15c will be located at the cross section of the receiving recess 7 defined by the first and second resilient portions 6c, 6d. In other words, the housing structure 6 is configured to receive the portion 15c of the medicament container structure 15 between the first and second resilient portions 6c, 6d. The first cross section area A1 of the receiving recess 7 (i.e. when the first and second resilient portions 6c, 6d are in their respective first position) is smaller than a cross section area A3 of the portion 15c of the medicament container structure 15. Thus, when the medicament container structure 15 reaches its final position within the receiving recess 7, the first and second resilient portions 6c, 6d are arranged to bear against the portion 15c of the medicament container structure 15. Hereby, the first and second resilient portions 6c, 6d are arranged in the receiving recess 7 to radially support the portion 15c of the medicament container structure 15.

Stated differently, the first and the second resilient portions 6c, 6d are two radially flexibly and radially inwards extending tabs 6c, 6d, configured to engage with the portion 15c of the medicament container structure 15. As previously described, each one of the first and second resilient portions 6c, 6d may extend both radially and longitudinally into the receiving recess 7. Each one of the tabs 6c, 6d may be inclined and present a decreasing ramp surface in the distal direction and may thereby flex radially outwards and engage with the portion 15c of the medicament container structure 15 upon insertion of the medicament container structure 15 into the receiving recess 7. The tabs 6c, 6d may be formed as ramps extending into the receiving recess, wherein each ramp surface decrease in the distal direction to facilitate interaction with the portion 15c of the medicament container structure 15. Stated differently, each one of the ramp surfaces is angled in the distal direction so that it faces towards the centre axis and towards the distal end 6b. Thus, a first end of each ramp surface is arranged closer to the centre axis compared to a second end of the same ramp surface, wherein each the ramp surface axially extends from the first end to the second end, and wherein the first end is arranged proximal of the second end. Each one of the ramp surfaces may e.g. form an angle relative to the centre axis of 45° or less.

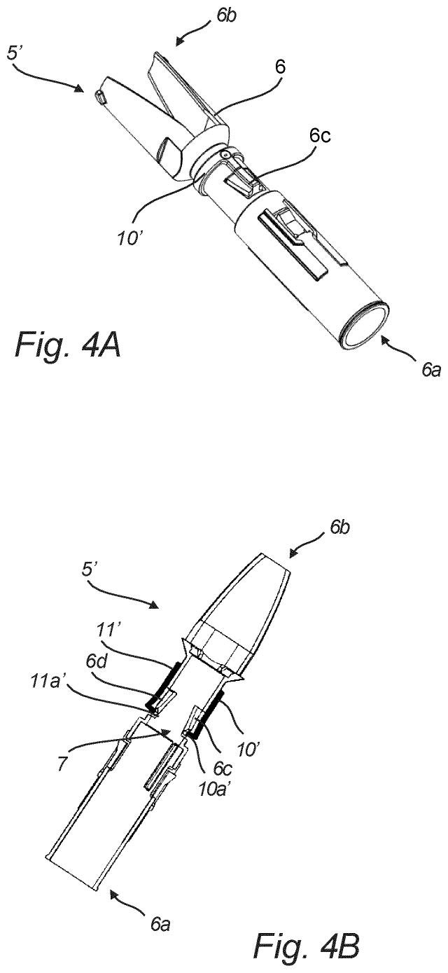

Turning to FIGS. 4A-4B, showing another example of a delivery member cover 5′. Correspondingly to the embodiment shown in FIG. 2, and FIGS. 3A-3B, delivery member cover 5′ of FIGS. 4A-4B is configured to cover at least the delivery member 22 as described with reference to FIG. 1, and comprises a housing structure 6 being the same, or similar, as the embodiment shown in FIG. 2, and FIGS. 3A-3B. Thus, the housing structure 6 comprises a proximal end 6a, a distal end 6b and a receiving recess 7 arranged between the proximal end 6a and the distal end 6b. Moreover, the housing structure 6 comprises a first and second resilient portion 6c, 6d. The receiving recess 7 is configured to receive at least a portion of the medicament container structure 15, in a corresponding manner as described with reference to FIGS. 3A-3B.

The deliver member cover 5′ comprises a thermoplastic elastomer 10′, 11′ arranged to at least partly cover a portion of the first and second resilient portions 6c, 6d. In more detail, a first thermoplastic elastomer 10′ is arranged to extend along a portion of the housing structure 6 and extend into the receiving recess 7 via an aperture in the outer surface of the housing structure 6, and to further cover the first resilient portion 6c on a portion facing the receiving recess 7. Hereby, the first resilient portion 6c comprises a contact surface 10a′ formed by the first thermoplastic elastomer 10′, which contact surface 10a′ is configured to be in contact with the portion 15c of the medicament container structure 15 upon insertion of the medicament container structure 15 into the receiving recess 7, which operation is not described here again. Correspondingly, the deliver member cover 5′ comprises a second thermoplastic elastomer 11′ arranged to extend along a portion of the housing structure 6 and extend into the receiving recess 7 via an aperture in the outer surface of the housing structure 6 opposite to the first thermoplastic elastomer 10′. The first and the second elastomers 10′, 11′ may alternatively form part of a single body. The second thermoplastic elastomer 11′ extend further inside the receiving recess 7 to cover the second resilient portion 6d on a portion facing the receiving recess 7. Hereby, the second resilient portion 6d comprises a contact surface 11a′ formed by the second thermoplastic elastomer 11′, which contact surface 11a′ is configured to be in contact with the portion 15c of the medicament container structure 15 upon insertion of the medicament container structure 15 into the receiving recess 7. Thus, when the medicament container structure 15 reaches its final position within the receiving recess 7, the contact surfaces 10a′, 11a′ of the first and second resilient portions 6c, 6d, respectively, are arranged to bear against the portion 15c of the medicament container structure 15. Alternatively, the respective thermoplastic elastomer 10′, 11′ forms the corresponding resilient portion 6c, 6d. That is, according to one embodiment, each one of the resilient portions 6c, 6d is formed by a respective thermoplastic elastomer 10′, 11′.

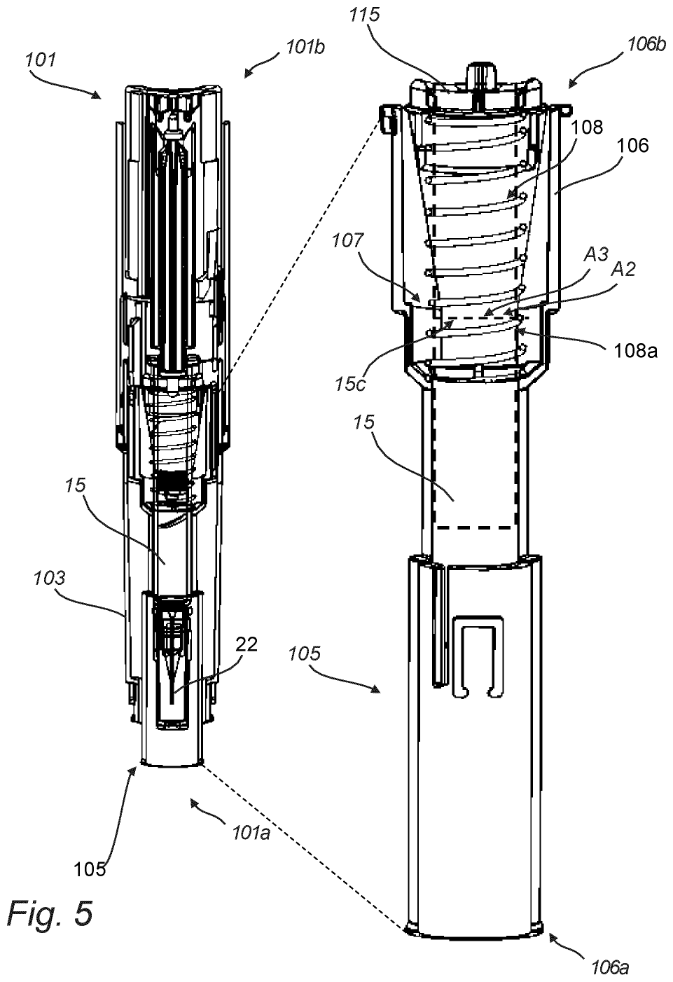

An alternative medicament delivery device 101 is shown in FIG. 5. The medicament delivery device 101 is configured to expel medicament from a medicament container structure 15 via a delivery member 22. The medicament container structure 15 may for example be a syringe, and the delivery member 22 a needle. The medicament delivery device 101 extends in the longitudinal direction from a proximal end 101a to a distal end 101b. The medicament delivery device 101 comprises a housing 103 and a delivery member cover 105, which in principle are operated in the same or a similar manner as the medicament deliver device 1 of FIG. 1, why mainly the differences are described in the following.

As shown in the enlarged view of FIG. 5, the delivery member cover 105 comprises a housing structure 106 having a proximal end 106a, a distal end 106b and a receiving recess 107 arranged between the proximal end 106a and the distal end 106b. The receiving recess 107 is configured to receive at least a portion of the medicament container structure 15. In the enlarged view of FIG. 5, the medicament container structure is only schematically shown as a dashed line box for increased comprehensibility. The delivery member cover 105 comprises a resilient portion 108 in the form a coil spring 108, or at least forming a part of the coil spring 108. The coil spring 108 is arranged inside the housing structure 106 such that the receiving recess 107 is arranged inside the coil spring 108.

The coil spring 108 is resilient, and flexibly arranged within the receiving recess 7. In the enlarged view of FIG. 5, the coil spring is arranged between a collar 115 to the medicament container structure 15, and an inner portion of the delivery member cover 105 arranged proximal of the collar 115. The coil spring 108 is configured to radially move within the receiving recess 7. That is, the radius of the coil spring 108 may be expanded upon an applied force in the radial direction. At least a first portion 108a of the coil spring 108 defines a cross section which is expandable from a first cross section area A1 to a second cross section area A2 (only cross section area A2 is indicated in the enlarged view of FIG. 5), wherein the second cross section area A2 is larger than the first cross section area A1. Stated differently, the first portion 108a of the coil spring 108 is defined by a diameter which is expandable from a first diameter to a second diameter (the second diameter defining the second cross section area A2 is indicated in the enlarged view of FIG. 5), wherein the second diameter is larger than the first diameter. Thus, the first portion 108a of the coil spring 108 is movable from a first position defined by the first cross section area, into a second position defined by the second cross section area.

The insertion of the medicament container structure 15 into the receiving recess 107, and pass the cross section of the receiving recess 107 inside the coil spring 108 being defined by first portion 108a, forces the first portion 108 to flex radially outwards into its second position. That is, the first portion 108a of the coil spring is configured to flex radially, and as the medicament container structure 15 moves at least partly pass the first portion 108a, the first portion 108a flex radially outwards. Thus, the first portions 108a is configured to be displaced from its first position to its second position upon insertion of the medicament container structure 15 into the receiving recess 107.

As the medicament container structure 15 reaches its final position within the receiving recess 107, a portion 15c will be located at the cross section of the receiving recess 107 defined by the first portion 108a of the coil spring 108. In other words, the delivery member cover 105 is configured to receive the portion 15c of the medicament container structure 15 inside of the first portion 108a of the coil spring 108. The first cross section area of the first portion 108a of the coil spring (i.e. prior insertion of the medicament container structure 15 into the receiving recess 107) is smaller than a cross section area A3 of the portion 15c of the medicament container structure 15. Thus, when the medicament container structure 15 reaches its final position within the receiving recess 107, the first portion 108a of the coil spring 108 is arranged to bear against the portion 15c of the medicament container structure 15. Hereby, the first portion 108a is arranged in the receiving recess 107 to radially support the portion 15c of the medicament container structure 15. Typically, the distal end of the coil spring 108 is radially supported by the collar 115. Hereby, the coil spring 108 is fixed in position and may thus advantageously radially support the portion 15c of the medicament container structure 15.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A delivery member cover of a medicament delivery device, the medicament delivery device comprising a medicament container structure having a delivery member for expelling a medicament, the delivery member cover being configured to cover at least the delivery member, the delivery member cover comprising:
a housing structure having a proximal end, a distal end, and a receiving recess arranged between the proximal end and the distal end, the receiving recess being configured to receive at least a portion of the medicament container structure; and
a resilient portion arranged inside the receiving recess, the resilient portion being configured to flex radially outwards to radially support said portion of the medicament container structure upon insertion of the medicament container structure into the receiving recess.

17. The delivery member cover according to claim 16, wherein the resilient portion is radially movable within the receiving recess between a first position in which the resilient portion defines a first cross section area of the receiving recess, and a second position in which the resilient portion defines a second cross section area of the receiving recess, wherein the second cross section area is larger than the first cross section area.

18. The delivery member cover according to claim 17, wherein the first cross section area in the first position of the resilient portion is smaller than a cross section area of said portion of the medicament container structure.

19. The delivery member cover according to claim 17, wherein the resilient portion is configured to be displaced from the first position to the second position upon insertion of the medicament container structure into the receiving recess.

20. The delivery member cover according to claim 16, wherein the resilient portion is configured to bear against said portion of the medicament container structure upon insertion of the medicament container structure into the receiving recess.

21. The delivery member cover according to claim 16, wherein the resilient portion comprises a contact surface configured to be in contact with said portion of the medicament container structure upon insertion of the medicament container structure into the receiving recess, wherein the contact surface comprises a thermoplastic elastomer.

22. The delivery member cover according to claim 16, wherein the resilient portion is arranged between the proximal end and the distal end of the housing structure.

23. The delivery member cover according to claim 16, wherein the resilient portion forms a part of the housing structure.

24. The delivery member cover according to claim 16, wherein the resilient portion extends from an outer surface of the housing structure into the receiving recess.

25. The delivery member cover according to claim 24, wherein the resilient portion is a first resilient portion and the delivery member cover further comprises a second resilient portion arranged on an opposite side of the housing structure compared to first resilient portion, wherein the housing structure is configured to receive said portion of the medicament container structure between the first and second resilient portions.

26. The delivery member cover according to claim 24, wherein the resilient portion is multiple resilient portions configured to receive said portion of the medicament container structure between the multiple resilient portions.

27. The delivery member cover according to claim 24, wherein the first resilient portion and/or the second resilient portion is a tab and/or the multiple resilient portions are tabs.

28. A medicament delivery device comprising a medicament container structure having a delivery member for expelling a medicament, and the delivery member cover according to claim 16.

29. The medicament delivery device according to claim 28, wherein the delivery member cover is configured to move axially relative to the delivery member from a delivery position in which the delivery member is uncovered for expelling the medicament, into a storage position in which the delivery member is covered by the delivery member cover.

30. The medicament delivery device according to claim 29, wherein the resilient portion of the delivery member cover is configured to radially support the medicament container structure while allowing axial movement of the medicament container structure within the delivery member cover.
